(19) United States
(12) Patent Application Publication
(10) Pub. No.: US 2014/0067000 A1
(43) Pub. Date: Mar. 6, 2014

(54) Implantable Medical Device Containing Fibers Formed from a Liquid Crystalline Polymer

(71) Applicant: Ticona LLC, Florence, KY (US)

(72) Inventors: Jill Higgins, Bloomfield Township, MI (US); Anthony Verrocchi, Covington, KY (US); Xinyu Zhao, Cincinnati, OH (US)

(73) Assignee: Ticona LLC, Florence, KY (US)

(21) Appl. No.: 14/018,545

(22) Filed: Sep. 5, 2013

Related U.S. Application Data
(60) Provisional application No. 61/697,368, filed on Sep. 6, 2012.

(51) Int. Cl. A61N 1/05

(52) U.S. Cl. CPC A61N 1/05
USPC 607/2; 607/116; 607/115

(57) ABSTRACT

An implantable medical device that employs one or more fibers formed from a polymer composition that contains at least one liquid crystalline polymer is provided. In addition to being highly inert and biocompatible, the rigid rod-like structure of liquid crystalline polymers can allow them to be particularly well suited for implantable devices. Apart from the benefits from the polymer itself, the fibers are formed in such a manner so that they are generally flexible, but yet still possess a sufficient degree of strength so that they can be effectively employed in implantable medical devices.

### RELATED APPLICATIONS

The present application claims priority to U.S. Provisional Application Ser. No. 61/697,368, filed on Sep. 6, 2012, which is incorporated by reference herein in its entirety.

### BACKGROUND OF THE INVENTION

Nerve stimulation has been proposed to treat a number of disorders pertaining to or mediated by one or more structures of the nervous system of the body, including epilepsy and other movement disorders, depression, anxiety disorders and other neuropsychiatric disorders, dementia, traumatic brain injury, head trauma, coma, migraine headache, obesity, eating disorders, sleep disorders, cardiac disorders (such as congestive heart failure and atrial fibrillation), hypertension, endocrine disorders (such as diabetes and hypoglycemia) and pain, among others. To deliver the desired neurostimulation signal to a target portion of a patient's body (e.g., a vagus nerve), an implantable medical device is generally employed that may contain one or more leads that terminate at their distal ends in one or more electrodes. The electrodes, in turn, are electrically coupled to tissue in the patient's body. For example, a number of electrodes may be attached to various points of a nerve or other tissue inside a human body for delivery of a neurostimulation signal to the central nervous system and/or peripheral nervous system.

Various electrode configurations may generally be employed in such devices. The electrode may, for example, be an extraneural electrode, such as a cuff electrode, flat interface nerve electrode (“FINE”), etc. Penetrating intrafascicular electrodes may also be employed, such as longitudinal intrafascicular electrodes (“LIFE”), transverse intrafascicular multichannel electrodes (“TIME”), and transcutaneuous electrical nerve stimulation (“TENS”). Most conventional electrodes for implantable devices employ metallic wires. For example, iridium and tungsten wires have been proposed for use as brain tissue penetrating electrodes. One of the problems with such wires, however, is that they are stiff and thus have difficulty adapting to the normal micromotion of the brain tissue (e.g., due to breathing, heart beating, etc.), which can lead to tissue damage. The use of more flexible electrodes is also problematic in that they may not always possess the desired conductivity, and it can be difficult to insert such electrodes into the cortex.

As such, a need currently exists for an improved electrode for an implantable medical device.

### SUMMARY OF THE INVENTION

In accordance with one embodiment of the present invention, an implantable medical device is disclosed that comprises one or more fibers, wherein the fibers are formed from a polymer composition that contains a liquid crystalline polymer.

In accordance with another embodiment, a method for delivering a stimulation signal to a target tissue in a body of a patient is also disclosed. The method comprises positioning an implantable medical device, such as described herein, in contact with the target tissue and passing an electrical stimulation signal through the electrode to the target tissue.

In accordance with yet another embodiment of the present invention, an electrode for a medical device is disclosed. The electrode comprises one or more fibers, wherein the fibers are formed from a polymer composition that contains a liquid crystalline polymer.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the specification, including reference to the accompanying figures, in which:

FIG. 1 is a perspective view of one embodiment an implantable medical device of the present invention;

FIG. 2 is a block diagram of the implantable medical device shown in FIG. 1;

FIG. 3 is a perspective view of another embodiment of an implantable medical device of the present invention.

### DETAILED DESCRIPTION

### Definitions

It is to be understood that the terminology used herein is for the purpose of describing particular embodiments only and is not intended to limit the scope of the present invention.

As used herein, the term “electrode” may encompass stimulation electrodes (e.g., electrodes for delivering an electrical signal to a tissue), sensing electrodes (e.g., electrodes for sensing a physiological indication), and/or electrodes that are capable of delivering a stimulation signal, as well as performing a sensing function.

As used herein, the term “implantable” generally refers to a device that is capable of or designed for being implanted in a patient, whether temporarily or permanently.

As used herein, the term “stimulation” or “stimulation signal” generally refers to the application of an electrical, mechanical, magnetic, electro-magnetic, photonic, audio and/or chemical signal to a target tissue in a patient's body. The signal is an exogenous signal that is distinct from the endogenous electrical, mechanical, and chemical activity (e.g., afferent and/or efferent electrical action potentials) generated by the patient's body and environment. In other words, the stimulation signal (whether electrical, mechanical, magnetic, electro-magnetic, photonic, audio, or chemical in nature) applied to the tissue is a signal applied from an artificial source, such as a neurostimulator.

As used herein, the term “therapeutic signal” generally refers to a stimulation signal delivered to a patient's body with the intent of treating a disorder by providing a modulating effect to the target tissue, such as neural tissue, The effect of a stimulation signal on electrical, chemical and/or mechanical activity in the target tissue is termed “modulation”; however, for simplicity, the terms “stimulating” and “modulating”, and variants thereof, are sometimes used interchangeably herein. In general, however, the delivery of an exogenous signal itself refers to “stimulation” of the target tissue, while the effects of that signal, if any, on the electrical, chemical and/or mechanical activity of the target tissue are properly referred to as “modulation.”

As used herein, the term “patient” generally encompasses both human and non-human animals. The term “non-human animals” may include vertebrates (e.g., mammals and non-mammals), such as non-human primates, mice, rats, sheep, dogs, cats, horses, cows, chickens, amphibians, fish, reptiles, etc.

As used herein, the term “liquid crystalline polymer” generally refers to a polymer that can possess a rod-like structure and exhibit a liquid crystalline behavior in its molten state (e.g., thermotropic nematic state). The polymer may possess a fully crystalline, semi-crystalline, or amorous-like structure under certain circumstances. For example, when dissolved in a solvent, the polymer may exhibit amorphous-like properties in that it becomes transparent and lacks an identifiable melting point. Yet, after heat treatment and solvent removal, the polymer may exhibit a highly-ordered liquid crystalline structure in which the molecules are aligned.

As used herein, the term “fibers” generally refers to elongate bodies in which the length dimension of is substantially greater than the transverse dimension. The cross-section of the fibers may vary widely, such as circular, flat or oblong in cross-section. The fibers may be in the form of individual staple fibers or filaments (continuous fibers), yarns, threads, strands, wires, ribbons, fabrics, etc. Yarns may include, for instance, multiple staple fibers that are braided or twisted together (“spun yarn”), filaments laid together without twist (“zero-twist yarn”), (3) filaments laid together with a degree of twist, (4) a single filament with or without twist (“monofilament”), etc. The yarn may or may not be texturized. When multiple fibers are employed, such as in a bundle or yarn, any number of fibers can be used, such as from about 10 to about 800, and in some embodiments, from about 50 to about 500.

### Test Methods

Melt Viscosity: The melt viscosity (Pa-s) may be determined in accordance with ISO Test No. 11443 at 360° C. and at a shear rate of 400 s⁻¹ and 1000 s⁻¹ using a Dynisco 7001 capillary rheometer. The rheometer orifice (die) may have a diameter of 1 mm, length of 20 mm, L/D ratio of 20.1, and an entrance angle of 180°. The diameter of the barrel was 9.55 mm±0.005 mm and the length of the rod may be 233.4 mm.

Intrinsic Viscosity: The intrinsic viscosity (“IV”) may be measured in accordance with ISO-1628-5 using a 50/50 (v/v) mixture of pentafluorophenol and hexafluoroisopropanol. Each sample may be prepared in duplicate by weighing about 0.02 grams into a 22 mL vial. 10 mL of pentafluorophenol (“PFP”) may be added to each vial and the solvent. The vials may be placed in a heating block set to 80° C. overnight. The following day 10 mL of hexafluoroisopropanol (“HFIP”) may be added to each vial. The final polymer concentration of each sample may be about 0.1%. The samples may be allowed to cool to room temperature and analyzed using a PolyVisc automatic viscometer.

Melting and Crystallization Temperatures: The melting temperature (“Tm”) and crystallization temperature (“Tc”) may be determined by differential scanning calorimetry (“DSC”) as is known in the art. The melting temperature is the differential scanning calorimetry (DSC) peak melt temperature as determined by ISO Test No. 11357. The crystallization temperature is determined from the cooling exotherm in the cooling cycle. Under the DSC procedure, samples may be heated and cooled at 20° C. per minute as stated in ISO Standard 10350 using DSC measurements conducted on a TA Q2000 Instrument.

It is to be understood by one of ordinary skill in the art that the present discussion is a description of exemplary embodiments only, and is not intended as limiting the broader aspects of the present invention.

Generally speaking, the present invention is directed to an implantable medical device that employs one or more fibers formed from a polymer composition that contains at least one liquid crystalline polymer. In addition to being highly inert and biocompatible, the rigid rod-like structure of liquid crystalline polymers can allow them to be particularly well suited for implantable devices. For example, due to the presence of a thermotropic nematic state in the melt, liquid crystalline polymers can exhibit a shear thinning behavior, which is particularly attractive in the fabrication of implantable medical device parts with intricate geometries (e.g., electrodes) because the polymers can flow well under heat and shear to uniformly fill complex parts at fast rates without excessive flashing or other detrimental processing issues. Apart from the benefits from the polymer itself, the fibers are formed in such a manner so that they are generally flexible, but yet still possess a sufficient degree of strength so that they can be effectively employed in implantable medical devices. This may be accomplished by selectively controlling both the manner in which the liquid crystalline polymer is formed (e.g., monomer selection and concentrations), as well as the process by which the fibers are formed.

Various embodiments of the present invention will now be described in further detail.

### I. Polymer Composition

While a wide variety of liquid crystalline polymers may generally be employed in the fibers, particularly suitable polymers are those that contain one or more aromatic ester repeating units, typically in an amount of from about 50 mol. % to about 99 mol. %, in some embodiments from about 55 mol. % to about 98 mol. %, and in some embodiments, from about 70 mol. % to about 95 mol. % of the polymer. The aromatic ester repeating units may be represented by the following Formula (I):

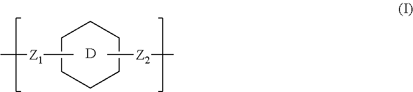

wherein,

ring D is a substituted or unsubstituted 6-membered aryl group (e.g., 1,4-phenylene or 1,3-phenylene), a substituted or unsubstituted 6-membered aryl group fused to a substituted or unsubstituted 5- or 6-membered aryl group (e.g., 2,6-naphthalene), or a substituted or unsubstituted 6-membered aryl group linked to a substituted or unsubstituted 5- or 6-membered aryl group (e.g., 4,4-biphenylene); and

Z₁ and Z₂ are independently O, C(O), NH, C(O)HN, or NHC(O), wherein at least one of Z₁ and Z₂ are C(O).

Examples of aromatic ester repeating units that are suitable for use in the present invention may include, for instance, aromatic dicarboxylic repeating units (Z₁ and Z₂ in Formula I are C(O)), aromatic hydroxycarboxylic repeating units (Z₁ is O and Z₂ is C(O) in Formula I), as well as various combinations thereof.

Aromatic hydroxycarboxylic repeating units, for instance, may be employed that are derived from aromatic hydroxycarboxylic acids, such as, 4-hydroxybenzoic acid; 4-hydroxy-4′-biphenylcarboxylic acid; 2-hydroxy-6-naphthoic acid; 2-hydroxy-5-naphthoic acid; 3-hydroxy-2-naphthoic acid; 2-hydroxy-3-naphthoic acid; 4′-hydroxyphenyl-4-benzoic acid; 3′-hydroxyphenyl-4-benzoic acid; 4′-hydroxyphenyl-3-benzoic acid, etc., as well as alkyl, alkoxy, aryl and halogen substituents thereof, and combination thereof. Particularly suitable aromatic hydroxycarboxylic acids are 4-hydroxybenzoic acid (“HBA”) and 2-hydroxy-6-naphthoic acid (“HNA”). When employed, repeating units derived from hydroxycarboxylic acids (e.g., HBA and/or HNA) typically constitute from about 40 mol. % or more, in some embodiments about 50 mol. % or more, and in some embodiments, from about 55 mol. % to 100 mol. % of the polymer.

Aromatic dicarboxylic repeating units may also be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl)ether, bis(4-carboxyphenyl)butane, bis(4-carboxyphenyl)ethane, bis(3-carboxyphenyl)ether, bis(3-carboxyphenyl)ethane, etc., as well as alkyl, alkoxy, aryl and halogen substituents thereof, and combinations thereof. Particularly suitable aromatic dicarboxylic acids may include, for instance, 2,6-naphthanlenedicarboxylic acid (“NDA”), terephthalic acid (“TA”), and isophthalic acid (“IA”). When employed, repeating units derived from aromatic dicarboxylic acids (e.g., NDA, IA, and/or TA) typically constitute from about 1 mol. % to about 50 mol. %, in some embodiments from about 2 mol. % to about 40 mol. %, and in some embodiments, from about 5 mol. % to about 30% of the polymer.

Other repeating units may also be employed in the polymer. In certain embodiments, for instance, repeating units may be employed that are derived from aromatic dials, such as hydroquinone, resorcinol, 2,6-dihydroxynaphthalene, 2,7-dihydroxynaphthalene, 1,6-dihydroxynaphthalene, 4,4′-dihydroxybiphenyl (or 4,4′-biphenol), 3,3′-dihydroxybiphenyl, 3,4′-dihydroxybiphenyl, 4,4′-dihydroxybiphenyl ether, bis(4-hydroxyphenyl)ethane, etc., as well as alkyl, alkoxy, aryl and halogen substituents thereof, and combinations thereof. Particularly suitable aromatic diols may include, for instance, hydroquinone (“HQ”) and 4,4′-biphenol (“BP”). When employed, repeating units derived from aromatic diols (e.g., HQ and/or BP) typically constitute from about 1 mol. % to about 30 mol. %, in some embodiments from about 2 mol. % to about 25 mol. %, and in some embodiments, from about 5 mol. % to about 20% of the polymer. Repeating units may also be employed, such as those derived from aromatic amides (e.g., acetaminophen (“APAP”)) and/or aromatic amines (e.g., 4-aminophenol (“AP”), 3-aminophenol, 1 ,4-phenylenediamine, 1,3-phenylenediamine, etc.). When employed, repeating units derived from aromatic amides (e.g., APAP) and/or aromatic amines (e.g., AP) typically constitute from about 0.1 mol. % to about 20 mol. %, in some embodiments from about 0.5 mol. % to about 15 mol. %, and in some embodiments, from about 1 mol. % to about 10% of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, diols, amides, amines, etc. Of course, in other embodiments, the polymer may be “wholly aromatic” in that it lacks repeating units derived from non-aromatic (e.g., aliphatic or cycloaliphatic) monomers.

In one particular embodiment, for example, the liquid crystalline polymer may contain repeating units derived from 4-hydroxybenzoic acid (“HBA”), terephthalic acid (“TA”), 2-hydroxy-6-naphthoic acid (“HNA”), 4,4′-biphenol (“BP”), as well as various other optional constituents. The repeating units derived from HBA may constitute from about 45 mol. % to about 75 mol. %, and in some embodiments, from about 50 mol. % to about 70% of the polymer; the repeating units derived from TA may constitute from about 10 mol. % to about 30 mol. %, and in some embodiments, from about 15 mol. % to about 25% of the polymer; the repeating units derived from HNA may constitute from about 1 mol. % to about 10 mol. %, and in some embodiments, from about 2 mol. % to about 6% of the polymer; and the repeating units derived from BP may constitute from about 5 mol. % to about 20 mol. %, and in some embodiments, from about 8 mol. % to about 15 mol. % of the polymer. In others embodiments, substantially all of the repeating units are formed from hydroxycarboxylic acids (e.g., HBA and/or HNA). In one embodiment, for instance, HBA may constitute from about 50 mol. % to about 90 mol. %, in some embodiments from about 60 mol. % to about 85 mol. %, and in some embodiments, from about 68 mol. % to about 78 mol. % of the polymer. Likewise, HNA may constitute from about 10 mol. % to about 50 mol. %, in some embodiments from about 15 mol. % to about 40 mol. %, and in some embodiments, from about 22 mol. % to about 32 mol. % of the polymer.

Regardless of its constituents, the liquid crystalline polymer may be prepared by introducing precursor monomer(s) used to form the backbone of the polymer (e.g., aromatic hydroxycarboxylic acid, aromatic dicarboxylic acid, aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, III, et al.; U.S. Pat. No. 6,114,492 to Linstid, III, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known in art. Acetylation may occur in in a separate reactor vessel, or it may occur in situ within the polymerization reactor vessel. When separate reactor vessels are employed, one or more of the monomers may be introduced to the acetylation reactor and subsequently transferred to the melt polymerization reactor. Likewise, one or more of the monomers may also be directly introduced to the reactor vessel without undergoing pre-acetylation. Acetylation may be accomplished by adding an acetylating agent (e.g., acetic anhydride) to one or more of the monomers. One particularly suitable technique for acetylating monomers may include, for instance, charging precursor monomers (e.g., 4-hydroxybenzoic acid and 2,6-hydroxynaphthoic acid) and acetic anhydride into a reactor and heating the mixture to acetylize a hydroxyl group of the monomers (e.g., forming acetoxy).

Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

After any optional acetylation is complete, the resulting composition may be melt-polymerized. Although not required, this is typically accomplished by transferring the acetylated monomer(s) to a separator reactor vessel for conducting a polycondensation reaction. If desired, one or more of the precursor monomers used to form the liquid crystalline polymer may be directly introduced to the melt polymerization reactor vessel without undergoing pre-acetylation. Other components may also be included within the reaction mixture to help facilitate polymerization. For instance, a catalyst may be optionally employed, such as metal salt catalysts (e.g., magnesium acetate, tin(I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. The catalyst is typically added to the acetylation reactor rather than the polymerization reactor, although this is by no means a requirement.

During melt polymerization, the reaction mixture is generally heated to an elevated temperature within the polymerization reactor vessel to initiate melt polycondensation of the reactants. Polycondensation may occur, for instance, within a temperature range of from about 210° C. to about 400° C., and in some embodiments, from about 250° C. to about 350° C. For instance, one suitable technique for forming the liquid crystalline polymer may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to a temperature of from about 210° C. to about 400° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a sectional pressure, such as within the range of from about 5 to about 30 pounds per square inch (“psi”), and in some embodiments, from about 10 to about 20 psi.

Following melt polymerization, the molten polymer may be discharged from the reactor, typically through an extrusion orifice fitted with a die of desired configuration, cooled, and collected. Commonly, the melt is discharged through a perforated die to form strands that are taken up in a water bath, pelletized and dried. The resin may also be in the form of a strand, granule, or powder. While unnecessary, it should also be understood that a subsequent solid phase polymerization may be conducted to further increase molecular weight. When carrying out solid-phase polymerization on a polymer obtained by melt polymerization, it is typically desired to select a method in which the polymer obtained by melt polymerization is solidified and then pulverized to form a powdery or flake-like polymer, followed by performing solid polymerization method, such as a heat treatment in a temperature range of 200° C. to 350° C. under an inert atmosphere (e.g., nitrogen).

Regardless of the particular method employed, the liquid crystalline polymer may have a relatively high number average molecular weight (Mₙ) of about 2,000 grams per mole or more, in some embodiments from about 4,000 grams per mole or more, and in some embodiments, from about 5,000 to about 500,000 grams per mole. The intrinsic viscosity of the polymer, which is generally proportional to molecular weight, may also be relatively high. For example, the intrinsic viscosity may be about 2 deciliters per gram (“dL/g”) or more, in some embodiments about 3 dL/g or more, in some embodiments from about 5 to about 20 dL/g, and in some embodiments from about 6 to about 15 dL/g. Intrinsic viscosity may be determined in accordance with ISO-1628-5 using a 50/50 (v/v) mixture of pentafluorophenol and hexafluoroisopropanol, as described in more detail below.

The melting point of the liquid crystalline polymer may likewise range from about 300° C. to about 400° C., in some embodiments from about 310° C. to about 390° C., and in some embodiments, from about 320° C. to about 380° C. Likewise, the crystallization temperature may range from about 250° C. to about 400° C., in some embodiments from about 260° C. to about 340° C., and in some embodiments, from about 280° C. to about 320° C. The melting and crystallization temperatures may be determined as is well known in the art using differential scanning calorimetry (“DSC”), such as determined by ISO Test No. 11357.

If desired, the polymer composition used to form the fibers may also contain one or more additives in conjunction with the liquid crystalline polymer. Examples of such additives may include, for instance, antimicrobials, pigments, antioxidants, stabilizers, surfactants, waxes, flow promoters, solid solvents, fillers, and other materials added to enhance properties and processibility. For example, a mineral filler material may be incorporated with the polymer composition, such as talc, calcium silicate, aluminum silicate, mica, diatomaceous earth, wollastonite, alumina, silica, titanium dioxide, calcium carbonate, and so forth. When employed, the additives may be combined together with the polymer using any of a variety of different techniques as is known in the art. In one particular embodiment, for example, the polymer and optional additives are melt processed as a mixture within an extruder to form the polymer composition.

### II. Fibers

Any of a variety of processes may be employed in the present invention to form fibers from the polymer composition, such as solution spinning, melt spinning, etc. For example, the composition may be extruded through a spinneret, quenched, and drawn into the vertical passage of a fiber draw unit. In one embodiment, for example, the composition may be supplied to an extruder (e.g., single screw) that includes a screw rotatably mounted and received within a barrel (e.g., cylindrical barrel), which may be heated. The composition is moved downstream from a feed end to a discharge end by forces exerted by rotation of the screw. The speed of the screw may be selected to help achieve the desired residence time, shear rate, melt processing temperature, etc. The extruder may employ one or multiple zones, at least one of which operates at a temperature in the range of about 20° C. to about 50° C. above the melting temperature of the polymer, such as at a temperature of from about 350° C. to about 450° C.

Any known method and apparatus may be employed to heat the polymer to form a melt. One suitable extrusion apparatus employs a contact melting method so that the melt residence time can be kept short and constant. The apparatus includes a heated surface against which a molded rod of the liquid crystalline polymer is pressed. The fluid stream of molten polymer is then introduced to the extrusion chamber inside of which is disposed a filter pack and a cylindrical orifice. After being passed through the filter pack, the polymer melt is extruded through the cylindrical orifice. After the fluid stream is extruded through the orifice, the polymer forms an elongated shaped article having the polymer molecules oriented substantially parallel to the flow direction. The extruded shaped article may then be drawn in the form of filaments and taken-up on a filament spool. The draw-down ratio may be selectively controlled in the present invention to achieve fibers having the desired properties. More particularly, the draw-down ratio is typically selected within a range of from about 4 to about 20, and in some embodiments, from about 5 to about 15. The draw-down ratio is the ratio of cross-sectional area of the orifice (A₁) to the cross-sectional area of the filament (A₂). This ratio is often also expressed as the ratio of the take-up speed of the filament (V₂) to the extrusion speed of the filament (V₁). Thus the draw-down ratio may be expressed in terms of the following equation:

Draw Down Ratio=A₁/A₂=V₂/V₁

If desired, the fibers may also be subjected to an additional heat treatment after they are extruded. Without intending to be limited by theory, it is believed that such a heat treatment can further increase the molecular weight and crystallinity of the polymer, thereby enhancing the mechanical strength of the fibers without significantly impacting their flexibility. For example, in certain embodiments, the fibers may be thermally treated at a temperature that is about 10° C. to about 30° C. below the melting temperature of the liquid crystalline polymer, at which temperature the fibers remains in a solid state. The heat treatment may, for example, occur at a temperature of from about 320° C. to about 370° C. Although not necessarily required, the heat treatment typically occurs in the presence of an inert gas, such as nitrogen, argon, helium, etc.

After spinning and optional heat treatment, the resulting fibers may have an average diameter in the range of from about 0.1 to 250 micrometers, in some embodiments from about 0,5 to about 100 micrometers, in some embodiments from about 1 to about 50 micrometers, and in some embodiments, from about 2 to 30 micrometers. Regardless of their size, however, the fibers may exhibit excellent strength. One parameter that is indicative of the relative strength of the fibers is “tenacity”, which indicates the tensile strength of a fiber expressed as force per unit linear density. For example, the fibers of the present invention may have a tenacity of from about 5 to about 50 grams-force (“g_{f}”) per denier, in some embodiments from about 7 to about 40 g_{f} per denier, and in some embodiments, from about 8 to about 30 g_{f} per denier. The fibers may, for example, have a denier (i.e., the unit of linear density equal to the mass in grams per 9000 meters of fiber) of from about 50 to about 3000, in some embodiments from about 200 to 3000, and in some embodiments, from about 650 to about 2000. The elongation at break may also be relatively high, such as about 1.0% or more, in some embodiments about 1.5% or more, and in some embodiments, from about 2% to about 5%. The fibers are also relatively flexible, yet possess a sufficient degree of stiffness to be readily employed in implantable medical devices. The degree of stiffness is generally represented by the “tensile modulus” of the fibers, which refers to the ratio of the change in tenacity (grams-force per denier) to the change in strain, expressed as a fraction of the original fiber length (in/in) (cm/cm). For example, the tensile modulus may range from about 250 to about 1000 gf/denier, in some embodiments from about 300 to about 800 g_{f}/denier, and in some embodiments, from about 400 to about 600 g_{f}/denier. The tenacity, elongation at break, and tensile modulus may be determined using a variety of techniques, one of which is described in ASTM D2256-10e1.

### III. Implantable Medical Device

Due to their unique combination of liquid crystalline behavior, strength, and flexibility, the fibers of the present invention may generally be employed in a wide variety of implantable medical devices. For example, the implantable medical device may be an active device, such as neurostimulators that are configured to provide a stimulation signal (e.g., therapeutic signal) to the central nervous system and/or peripheral nervous system, cardiac pacemakers or defibrillators, etc. Electrical neurostimulation may be provided by implanting an electrical device underneath the patient's skin and delivering an electrical signal to a nerve, such as a cranial nerve. In one embodiment, the electrical neurostimulation involves sensing or detecting a body parameter, with the electrical signal being delivered in response to the sensed body parameter. This type of stimulation is generally referred to as “active”, “feedback”, or “triggered” stimulation. In another embodiment, the system may operate without sensing or detecting a body parameter once the patient has been diagnosed with a medical condition that may be treated by neurostimulation. In this case, the system may apply a series of electrical pulses to the nerve (e.g., a cranial nerve such as a vagus nerve) periodically, intermittently, or continuously throughout the day, or over another predetermined time interval. This type of stimulation is generally referred to as “passive”, “non-feedback”, or “prophylactic” stimulation. The electrical signal may be applied by an implantable medical device that is implanted within the patient's body. In another alternative embodiment, the signal may be generated by an external pulse generator outside the patient's body, coupled by an RF or wireless link to an implanted electrode.

Regardless of its particular nature, the implantable medical device may contain an electrode that is electrically coupled to a portion of the patient's body (e.g., tissue, nerves, etc.) for delivery of a stimulation signal to the central nervous system and/or peripheral nervous system. If desired, the fibers of the present invention may be employed in such an electrode. The fibers may be electrically non-conductive and thus “passive” to the extent that they are present for purposes other than the delivery of the stimulation signal, such as providing mechanical support, facilitating tissue penetration, etc. Likewise, the fibers may also be electrically conductive and thus “active” to the extent that they help provide the electrical pathway for delivery of the stimulation signal. When the fibers are employed as an active component of the electrode, the desired degree of electrical conductivity may be accomplished using any of a variety of known techniques. For example, a conductive coating may be deposited onto the fibers by sputtering, lithography, lamination, electroless deposition, chemical vapor deposition, etc. One particularly suitable technique is cylindrical lithography, such as described in U.S. Pat. Nos. 5,106,455, 5,269,882, and 5,273,622 to Jacobsen et al. The conductive coating may include a variety of conductive materials, such as platinum, iridium, tungsten, gold, rhodium, palladium, etc.

Various electrode configurations may generally be employed. The electrode may, for example, be an extraneural electrode, such as a cuff electrode that encircles the targeted nerve and contains a number of sites on its inner surface that face the nerve. Another suitable extraneural electrode is a flat interface nerve electrode (“FINE”) which, like a cuff electrode, also encircles the nerve but nonetheless reshapes it into a more electrically favorable geometry. Penetrating intrafascicular electrodes may also be employed that are more invasive than extraneural electrodes in that they placed in contact with a distinct fascicle in the nerve. Examples of such penetrating electrodes include, for instance, longitudinal intrafascicular electrodes (“LIFE”) and transverse intrafascicular multichannel electrodes (“TIME”). Moreover, other electrodes, such as spinal cord electrodes, deep brain stimulation (DBS) electrodes, electrodes for muscle stimulation, and electrodes for stimulation of organs or even bones could also be modified.

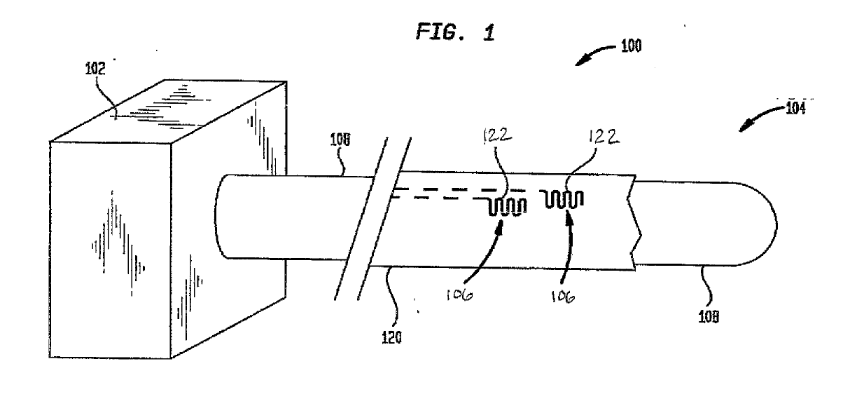

Referring to FIG. 1, for example, one embodiment of a neurostimulator 100 is shown that is configured to penetrate tissue (e.g., brain tissue). As shown, the neurostimulator 100 contains an implantable, hermetically sealed electronics module 102 and an electrode assembly 104. In the illustrative embodiment, the electrode assembly 104 contains an electrically non-conductive support structure 108 mechanically coupled to the electronics module 102. Disposed on the surface of the support structure 108 is an electrically non-conductive needle-piercable base 120. Electrodes 106 are connected (e.g., stitched, adhered, welded, etc.) to the base 120 that contain electrically conductive fibers 122 that extend to the electronics module 102. If desired, the electrically conductive fibers 122 may be formed in accordance with the present invention.

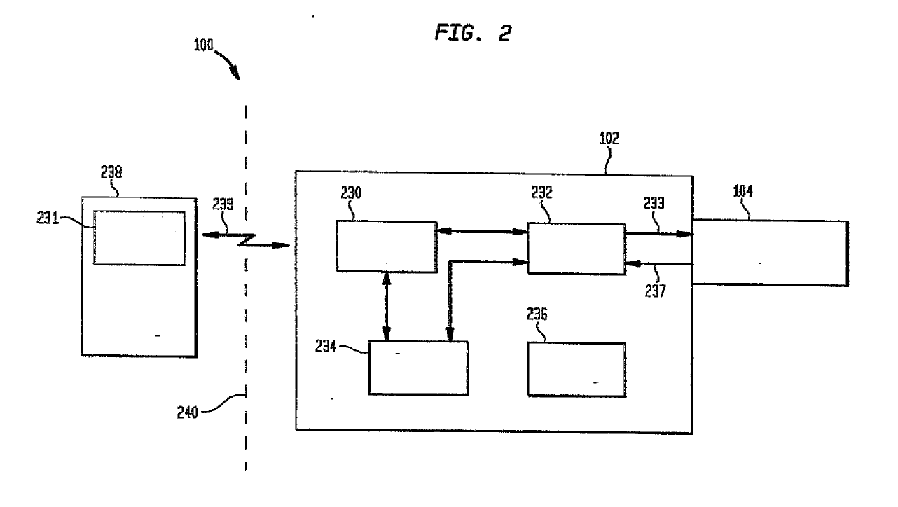

FIG. 2 is a functional block diagram illustrating one exemplary arrangement of the electronics module 102. In the illustrated embodiment, the electronics module 102 is implanted under a patient's skin/tissue 240, and cooperates with an external device 238 that contains an external transceiver unit 231 forming a bi-directional transcutaneous communication link 239 with an internal transceiver unit 230 of the electronics module 102. The transcutaneous communication link 239 may be used by external device 238 to transmit power and/or data to the electronics module 102. Similarly, the transcutaneous communication link 239 may be used by the electronics module 102 to transmit data to the external device 238. The transceiver units 230 and 231 include a collection of one or more components configured to receive and/or transfer power and/or data. The units 230 and 231 may, for instance, contain a coil for a magnetic inductive arrangement, a capacitive plate, or any other suitable arrangement. As such, various types of transcutaneous communication, such as infrared (IR), electromagnetic, capacitive and inductive transfer, may be used to transfer the power and/or data between the external device 238 and the electronics module 102. The electronics module 102 also includes a stimulator unit 232 that generates electrical stimulation signals 233. The electrical stimulation signals 233 may be delivered to a patient's tissue through the electrodes 106 (FIG. 1) of the electrode assembly 104 described above. The stimulator unit 232 may generate electrical stimulation signals 233 based on, for example, data received from the external device 238, signals received from a control module 234, in a pre-determined or pre-programmed pattern, etc. The electrodes may also be configured to record or monitor the physiological response of a patient's tissue. In such embodiments, signals 237 representing the recorded response may be provided to the stimulator unit 232 for forwarding to the control module 234, or to the external device 238 via the transcutaneous communication link 239.

In the embodiment shown in FIGS. 1-2, the neurostimulator 100 is an implantable medical device that is capable of operating, at least for a period of time, without the need for the external device 238. Therefore, the electronics module 102 may contain a rechargeable power source 236 that stores power received from the external device 238. The power source may include, for example, a rechargeable battery. During operation of the neurostimulator 100, the power stored by the power source is distributed to the various other components of the electronics module 102 as needed. For ease of illustration, electrical connections between the power source 236 and the other components of the electronics module 102 have been omitted, FIG. 2 shows the power source 236 within electronics module 102, but in other embodiments, it may be disposed in a separate implanted location. It should also be understood that the implanted power source may be omitted. For example, the neurostimulator 100 may be configured to operate based on power continually or periodically provided by an inductive link.

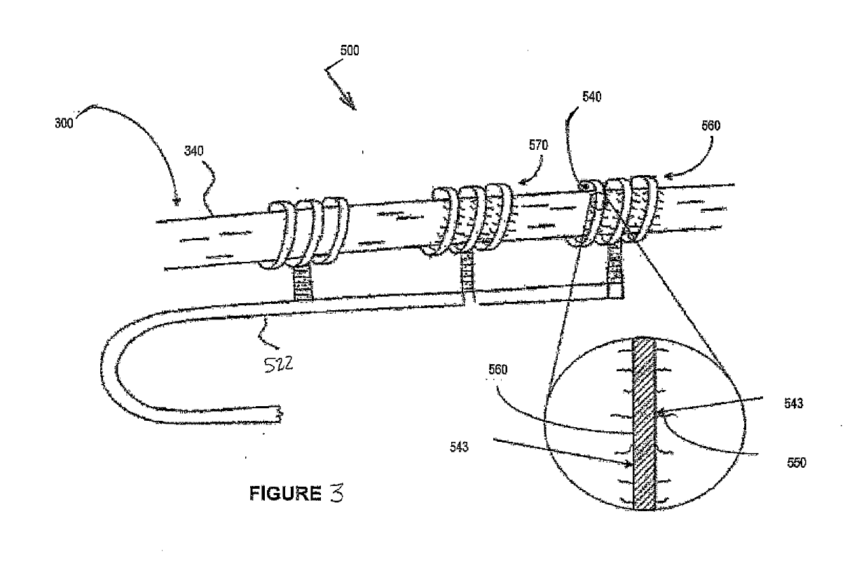

The electrode of the neurostimulator referenced above is designed to penetrate tissue. As noted, however, various other electrode configurations may also be employed in the present invention. Referring to FIG. 3, for example, one embodiment of a cuff electrode assembly 500 is shown that is designed to surround and penetrate an outer surface 340 of a nerve bundle 300. The electrode assembly 500 includes a plurality of fibers 550 protruding radially inwardly toward the nerve bundle 300 from an inner surface 540 and/or side surfaces 543 of a cathode 560 and/or anode 570. The fibers 550 provide for an electrical path from the implantable device (not shown) through a lead assembly 522. For example, the electrode assembly 500 may provide a path from the fibers 550 and inner surface 540 of the cathode 560, through the outer surface 340 of the nerve bundle 300 and into the inner portion thereof, through the nerve tissue and into the fibers 550, and to the inner surface 540 of the anode 570. If desired, the fibers 550 may be formed in accordance with the present invention.

Regardless of its configuration, a modulating effect may be imparted by the stimulation signal received by the target tissue from the electrode. The effect may be excitatory or inhibitory, and may potentiate acute and/or long-term changes in electrical, chemical and/or mechanical activity. For example, the “modulating” effect to a target neural tissue may have one more of the following effects: (a) initiation of an action potential (afferent and/or efferent action potentials); (b) inhibition or blocking of the conduction of action potentials, whether endogenously or exogenously induced, including hyperpolarizing and/or collision blocking; (c) affecting changes in neurotransmitter/neuromodulator release or uptake; and (d) changes in neuro-plasticity or neurogenesis of brain tissue. The modulating effect may be used to help treat a variety of different conditions, such as epilepsy and other movement disorders, depression, anxiety disorders and other neuropsychiatric disorders, dementia, traumatic brain injury, head trauma, coma, migraine headache, obesity, eating disorders, sleep disorders, cardiac disorders (such as congestive heart failure and atrial fibrillation), hypertension, endocrine disorders (such as diabetes and hypoglycemia) and pain, among others.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. An implantable medical device comprising one or more fibers, wherein the fibers are formed from a polymer composition that contains a liquid crystalline polymer.

2. The implantable medical device of claim 1, wherein the liquid crystalline polymer contains from about 50 mol. % to about 99 mol. % of aromatic ester repeating units having the following Formula (I): 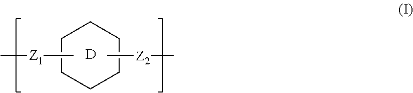
wherein,
ring D is a substituted or unsubstituted 6-membered aryl group, a substituted or unsubstituted 6-membered aryl group fused to a substituted or unsubstituted 5- or 6-membered aryl group, or a substituted or unsubstituted 6-membered aryl group linked to a substituted or unsubstituted 5- or 6-membered aryl group; and
Z₁ and Z₂ are independently O, C(O), NH, C(O)HN, or NHC(O), wherein at least one of Z₁ and Z₂ are C(O).

3. The implantable medical device of claim 2, wherein Ring D is 1,4-phenylene, 2,6-naphthalene, 4,4-biphenylene, or a combination thereof.

4. The implantable medical device of claim 2, wherein the aromatic ester repeating units are derived from an aromatic dicarboxylic acid that includes terephthalic acid, isophthalic acid, 2,6-naphthalenedicarboxylic acid, or a combination thereof.

5. The implantable medical device of claim 2, wherein the aromatic ester repeating units are derived from an aromatic hydroxycarboxylic acid that includes 4-hydroxybenzoic acid, 2-hydroxy-6-naphthoic acid, or a combination thereof.

6. The implantable medical device of claim 2, wherein the liquid crystalline polymer further comprises one or more repeating units derived from an aromatic diol, aromatic amide, aromatic amine, or a combination thereof.

7. The implantable medical device of claim 1, wherein the liquid crystalline polymer is wholly aromatic.

8. The implantable medical device of claim 1, wherein the liquid crystalline polymer has a melting temperature of from about 300° C. to about 400° C.

9. The implantable medical device of claim 1, wherein the fibers have an average diameter in the range of from about 0.1 to 250 micrometers.

10. The implantable medical device of claim 1, wherein the fibers are in the form of filaments.

11. The implantable medical device of claim 1, wherein the fibers have a tenacity of from about 5 to about 50 grams-force per denier, an elongation at break of about 1.0% or more, and/or a tensile modulus of from about 250 to about 1000 grams-force per denier.

12. The implantable medical device of claim 1, wherein the fibers are electrically conductive.

13. The implantable medical device of claim 12, wherein the fibers contain a conductive coating.

14. The implantable medical device of claim 1, wherein the fibers are electrically non-conductive.

15. The implantable medical device of claim 1, wherein the device is a neurostimulator that is configured to deliver a stimulation signal.

16. The implantable medical device of claim 19, wherein the neurostimulator contains an electrode, the electrode comprising the one or more fibers.

17. The implantable medical device of claim 20, wherein the electrode is an extraneural electrode or a penetrating intrafascicular electrode.

18. A method for delivering a stimulation signal to a target tissue in a body of a patient, the method comprising:
positioning the implantable medical device of claim 1 in contact with the target tissue; and
passing an electrical stimulation signal through the electrode to the target tissue.

19. An electrode for a medical device, the electrode comprising one or more fibers, wherein the fibers are formed from a polymer composition that contains a liquid crystalline polymer.

20. The electrode of claim 19, wherein the liquid crystalline polymer contains from about 50 mol. % to about 99 mol. % of aromatic ester repeating units having the following Formula (I): 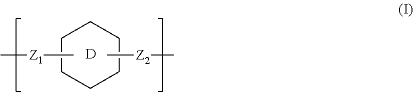
wherein,
ring D is a substituted or unsubstituted 6-membered aryl group, a substituted or unsubstituted 6-membered aryl group fused to a substituted or unsubstituted 5- or 6-membered aryl group, or a substituted or unsubstituted 6-membered aryl group linked to a substituted or unsubstituted 5- or 6-membered aryl group; and
Z₁ and Z₂ are independently O, C(O), NH, C(O)HN, or NHC(O), wherein at least one of Z₁ and Z₂ are C(O).

21. The electrode of claim 19, wherein Ring D is 1,4-phenylene, 2,6-naphthalene, 4,4-biphenylene, or a combination thereof.

22. The electrode of claim 19, wherein the aromatic ester repeating units are derived from an aromatic dicarboxylic acid that includes terephthalic acid, isophthalic acid, 2,6-naphthalenedicarboxylic acid, or a combination thereof.

23. The electrode of claim 19, wherein the aromatic ester repeating units are derived from an aromatic hydroxycarboxylic acid that includes 4-hydroxybenzoic acid, 2-hydroxy-6-naphthoic acid, or a combination thereof.

24. The electrode of claim 19, wherein the liquid crystalline polymer further comprises one or more repeating units derived from an aromatic dial, aromatic amide, aromatic amine, or a combination thereof.

25. The electrode of claim 19, wherein the liquid crystalline polymer is wholly aromatic.

26. The electrode of claim 19, wherein the fibers contain a conductive coating.

27. The electrode of claim 19, wherein the electrode is an extraneural electrode or a penetrating intrafascicular electrode.
